(19) United States
(12) Patent Application Publication
(10) Pub. No.: US 2026/0192842 A1
(43) Pub. Date: Jul. 9, 2026

(54) TROLLEY POWER CABLE GUIDE

(71) Applicant: DJO, LLC, Carlsbad, CA (US)

(72) Inventors: Nicolas FONTAINE, Bottens (CH); Cédric GIRARDIN, Ogens (CH)

(21) Appl. No.: 19/134,321

(22) PCT Filed: Nov. 30, 2023
(86) PCT No.: PCT/US2023/081854

§ 371 Date: May 30, 2025

Related U.S. Application Data
(60) Provisional application No. 63/386,138, filed on Dec. 5, 2022.

(51) Int. Cl. B62B 5/00; A61B 50/13; H02G 11/02

(52) U.S. Cl. CPC B62B 5/00; A61B 50/13; H02G 11/02

(57) ABSTRACT

A system for guiding a/some cable(s) on a trolley. This system uses at least one guide to route the cable(s) of a trolley above the trolley's wheel. This prevents the cable(s) from interfering with the mobility of the trolley. Improved mobility includes the ability of the trolley to rotate with an increased range. Guides can be located on the chassis, the base, and the legs of the trolley. The route of the cable(s) can be adjusted to run along one of multiple legs using guides on each leg.

### INCORPORATION BY REFERENCE TO ANY PRIORITY APPLICATIONS

This application claims the benefit of U.S. Provisional Application No. 63/386,138, filed Dec. 5, 2022, which is incorporated by reference herein in its entirety. Any and all applications for which a foreign or domestic priority claim is identified in the Application Data Sheet as filed with the present application are hereby incorporated by reference under 37 CFR 1.57 for all purposes and for all that they contain.

### BACKGROUND

### Field

The present application relates to systems for guiding cables, for example, cable management systems for movable trolleys, carts, or stands used in medical or commercial facilities.

### SUMMARY

Various embodiments described herein relate to systems, devices, and methods for guiding or routing a power cable, or other type of cable, associated with electronic or other equipment that is mounted on a trolley, cart, or moveable stand. Although the cable routing systems and methods are not limited to the context of the medical field, medical equipment mounted on trolleys provides a particular example that can benefit from the cable routing concepts described herein. For example, in hospitals, doctors' offices, and other medical facilities, medical equipment mounted on moveable carts or trolleys (e.g., carts or trolleys that include wheels) is common. Mounting the medical equipment on trolleys allows the equipment to be moved to various desired positions during use. However, the medical equipment is often connected to one or more cables, such as power cables, network cables, cables connecting to various sensors, cables connecting to equipment, and the like. To allow movement of the trolley while the equipment maintains its connection, the cables are of various lengths and are typically positioned on a floor on which the trolley's wheels travel when the trolley is moved. During movement of the trolley, these cables can interfere with the wheels of the trolley which can make movement of the trolley difficult or even cause the trolley to tip over. The systems, methods, and devices for guiding cables associated with such trolleys as described herein can resolve or reduce these problems.

In some examples, the systems, methods, and devices for guiding cables associated with trolley include one or more guides or clips positioned on the trolley and configured to secure a cable to the trolley. The guides or clips can be arranged on the trolley to guide the cable from where the cable attaches to the equipment positioned on the cart to a position generally close to the ground but above or over one of the wheels of the trolley. In this way, the cable is maintained by the guides or clips in proximity to the cart and guided to an exit point generally close to the ground but above or over a wheel of the cart. With the cable secured by the guides or clips, the trolley can now be moved without the cable interfering with the wheels of the cart. This is especially true for small movements of the cart, such as small translations or rotations of the cart, but may help with larger movements of the cart as well.

In one aspect, a trolley configured with one or more guides for routing a cable is described. The trolley can include a base configured to support the trolley, a chassis fixed above the base of the trolley, a cable extending from the chassis of the trolley, one or more legs extending from the base of the trolley, and one or more wheels fixed beneath the one or more legs of the trolley. In some embodiments, the systems, devices, and methods herein comprise at least one guide fixed to one of the one or more legs of the trolley, wherein the at least one guide is configured to route the cable above one of the one or more wheels of the trolley.

In some embodiments, the trolley comprises a medical trolley. In some embodiments, the at least one guide comprises a substantially planar body extending from the trolley and a triangular body extending approximately perpendicularly from a distal end of the substantially planar body. In some embodiments, the at least one guide comprises a substantially planar body extending from the trolley and a semi-circular body extending approximately perpendicularly from a distal end of the substantially planar body. In some embodiments, the at least one guide comprises a substantially curved body extending from the trolley and a semi-circular body extending approximately perpendicularly from a distal end of the substantially curved body. In some embodiments, the at least one guide is able to be removed and repositioned on the trolley. In some embodiments, the at least one guide is located on one of the one or more legs of the trolley. In some embodiments, the trolley has at least two legs, at least one guide is located on each of the at least two legs of the trolley, and the cable can be repositioned from one of the at least two legs of the trolley to another of the at least two legs of the trolley. In some embodiments, the systems, devices, and methods herein further comprise a guide having an aperture facing downward on the base of the trolley, two guides having apertures facing upward on one of the one or more legs of the trolley, and one guide having an aperture facing downward on one of the one or more legs of the trolley. In some embodiments, the systems, devices, and methods herein further comprise a guide on the chassis of the trolley.

In some embodiments, the systems, devices, and methods herein comprise at least one guide fixed to a trolley, wherein the at least one guide is configured such that positioning the cable of the trolley along the at least one guide routes the cable above a wheel of the trolley.

In some embodiments, the trolley comprises a medical trolley. In some embodiments, the at least one guide comprises a substantially planar body extending from the trolley and a triangular body extending approximately perpendicularly from a distal end of the substantially planar body. In some embodiments, the at least one guide comprises a substantially planar body extending from the trolley and a semi-circular body extending approximately perpendicularly from a distal end of the substantially planar body. In some embodiments, the at least one guide comprises a substantially curved body extending from the trolley and a semi-circular body extending approximately perpendicularly from a distal end of the substantially curved body. In some embodiments, the at least one guide is able to be removed and repositioned on the trolley. In some embodiments, the at least one guide is located on one or more legs of the trolley. In some embodiments, the trolley has at least two legs, at least one guide is located on each of the at least two legs of the trolley, and the cable can be repositioned from one of the at least two legs of the trolley to another of the at least two legs of the trolley. In some embodiments, the systems, devices, and methods herein further comprise a guide having an aperture facing downward on a base of the trolley, two guides having apertures facing upward on a leg of the trolley, and one guide having an aperture facing downward on the leg of the trolley. In some embodiments, the systems, devices, and methods herein further comprise guide on a chassis of the trolley.

Certain aspects of the systems, devices, and methods described herein are defined by the independent claims. The dependent claims include optional features of some embodiments of the systems, devices, and methods described herein. The systems, methods, and devices described herein each have several aspects, no single one of which is solely responsible for its desirable attributes. Without limiting the scope of this disclosure, several non-limiting features will now be discussed briefly.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

In the drawings, which are not necessarily to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an embodiment of a trolley with a system for guiding a cable;

FIG. 2 is a detailed view of a base and leg of the trolley of FIG. 1;

FIG. 3 illustrates an embodiment of a planar, triangular guide used in the systems, devices, and methods described herein;

FIG. 4 illustrates an embodiment of a planar, semi-circular guide used in the systems, devices, and methods described herein;

FIG. 5 illustrates an embodiment of a curved, semi-circular guide used in the systems, devices, and methods described herein;

FIG. 6A-D illustrate embodiments of the rotation of a trolley using (C, D), and not using (A, B) the systems, devices, and methods described herein;

FIG. 7 illustrates an embodiment of a possible cable route according to the systems, devices, and methods described herein;

FIG. 8 illustrates an embodiment of an alternate possible cable route using systems, devices, and methods described herein;

FIG. 9A illustrates an embodiment of a front view of a trolley; and

FIG. 9B illustrates an embodiment of a back view of a trolley.

### DETAILED DESCRIPTION

A better understanding of different embodiments of the systems, devices, and methods described herein may be had from the following description, read in conjunction with the accompanying drawings, in which like reference characters refer to like elements.

Embodiments of a system for guiding a cable are disclosed herein. Although certain illustrative embodiments are shown in the drawings and will be described below in detail, the application is not limited to these embodiments. However, there is no intention to limit the disclosure to the specific embodiments disclosed. On the contrary, the disclosure is intended to cover all modifications, alternative constructions, combinations, and equivalents falling within the spirit and scope of the disclosure.

In some embodiments, the systems, devices, and methods described herein are related to guiding a cable of a trolley. In some embodiments, the trolley comprises a medical trolley. In some embodiments, a medical trolley can mean a movable cart used in hospitals, doctors' offices, clinics, or the like. In some embodiments, the trolley is able to move via wheels fixed to the bottom of the trolley. In some embodiments, the wheels of the trolley are fixed beneath legs of the trolley. In some embodiments, a medical trolley can mean a movable cart used for medical purposes. In some embodiments, the trolley comprises a commercial trolley. In some embodiments, a commercial trolley can mean a movable cart used in a commercial office. In some embodiments, a commercial trolley can mean a movable cart used for commercial purposes.

With respect to devices currently on the market, trolleys often have problems with mobility because their cables can interfere with the movement of their wheels. Contact between the trolley wheel and the cable can cause the trolley to stop, can cause the cable to get tangled, and/or can cause the cable to become unplugged. Cables on current trolleys often extend from the center of the chassis of the trolley to the floor. Because cables on current trolleys extend down from the center of the chassis of the trolley, cables sometimes touch the floor between wheels of the trolley. In some embodiments, systems, devices, and methods described herein guide the cable to avoid contact with the trolley wheel. In some embodiments, systems, devices, and methods described herein guide the cable along the trolley leg and above the trolley wheel.

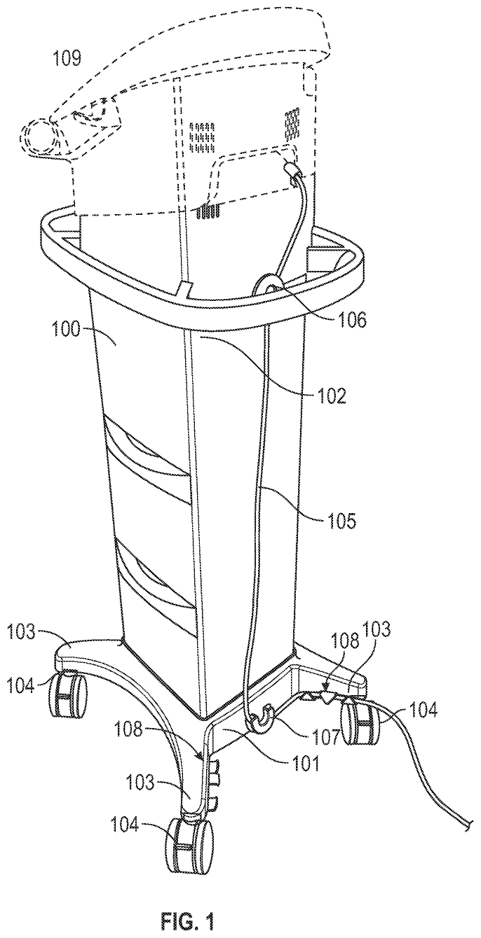

FIG. 1 illustrates an example of an embodiment of a cable guiding system on a trolley 100. The trolley 100 can include: a base 101, a chassis 102 fixed to the top of the base 101, legs 103 extending from the base 101, wheels 104 fixed to the bottom of the legs 103, a cable 105 extending from the chassis 102, a guide 106 on the chassis 102, a guide 107 on the base 101, and guides 108 on the legs 103. The cable 105 can be routed through the guide 106, the guide 107, and/or the guides 108. Routing the cable 105 through the guides 106, 107, 108 can prevent the cable from interfering with the movement of the trolley 100. For example, the guides 106, 107, 108 can prevent the cable from contacting the wheels 104 of the trolley 100.

“Trolley” as used herein is a broad term that refers to a movable device (e.g., a cart or trolley) that typically includes wheels and is configured to carry or move objects or equipment. In some examples, the trolley 100 can be a medical trolley, for example, a movable cart used in hospitals or for medical related purposes. In some embodiments, the trolley 100 can be a commercial trolley. A commercial trolley can mean a movable cart used in a commercial office or for commercial purposes.

The base 101 of the trolley 100 can support the trolley 100. The base 101 of the trolley 100 can be positioned below the chassis 102 of the trolley 100. The base 101 of the trolley 100 can be fixed to the legs 103 of the trolley. The base 101 of the trolley 100 can include wheels 104 for movement of the trolley 100.

The chassis 102 can be positioned above the base of the trolley 100. In some embodiments, the chassis 102 is fixed to the top of the base 101 of the trolley 100. In some embodiments, the cable 105 extends from the chassis 102 of the trolley 100.

In some embodiments, the system can include one leg 103 of a trolley 100. In some embodiments, the system can include two legs 103 of a trolley 100. In some embodiments, the system can include three legs 103 of a trolley 100. In some embodiments, the system can include four legs 103 of a trolley 100. In some embodiments, the system can include five legs 103 of a trolley 100. In some embodiments, the system can include six legs 103 of a trolley 100. In some embodiments, the system can include seven legs 103 of a trolley 100. In some embodiments, the system can include eight legs 103 of a trolley 100. In some embodiments, the system can include more than eight legs 103 of a trolley 100. In some embodiments, the legs 103 extend from the base 101 of the trolley 100. In some embodiments, the legs 103 are fixed to the wheels of the trolley 100.

In some embodiments, the system can include one wheel 104 of a trolley 100. In some embodiments, the system can include two wheels 104 of a trolley 100. In some embodiments, the system can include three wheels 104 of a trolley 100. In some embodiments, the system can include four wheels 104 of a trolley 100. In some embodiments, the system can include five wheels 104 of a trolley 100. In some embodiments, the system can include six wheels 104 of a trolley 100. In some embodiments, the system can include seven wheels 104 of a trolley 100. In some embodiments, the system can include eight wheels 104 of a trolley 100. In some embodiments, the system can include more than eight wheels 104 of a trolley 100. In some embodiments, the system can include two wheels 104 of a trolley 100. In some embodiments, the wheels 104 are fixed beneath the legs 103 of the trolley 100. In some embodiments, the wheels 104 are fixed beneath the base 101 of the trolley 100.

In some embodiments, the system can include a cable 105. In some embodiments, the cable 105 can be a power cable. In some embodiments, the cable 105 can be a network cable. In some embodiments, the cable 105 is a cable connecting to a sensor. In some embodiments, the cable 105 can be a cord. In some embodiments, the cable 105 extends from the chassis 102 of the trolley 100. With respect to devices currently on the market, cables on trolleys extend from the chassis to the ground with no support or means of redirection. In some embodiments, the cable 105 extends from the base 101 of the trolley 100. Certain trolleys have a decreased range of motion because their wheels bump into their cables as they move or rotate. Advantageously, this system can prevent the cable 105 from interfering with the wheels 104 of the trolley 100. In some embodiments, the cable 105 is raised above a wheel 104 of the trolley 100 using a guide 108 on the leg 103 of the trolley 100. In some embodiments, the cable 105 is approximately one twentieth of an inch wide. In some embodiments, the cable 105 is approximately one fifteenth of an inch wide. In some embodiments, the cable 105 is approximately one tenth of an inch wide. In an example of a preferred embodiment, the cable 105 is approximately four twenty-fifths of an inch wide. In another example of a preferred embodiment, the cable 105 is approximately one fifth of an inch wide. In another example of a preferred embodiment, the cable 105 is approximately seven twenty-fifths of an inch wide. In some embodiments, the cable 105 is approximately three eighths of an inch wide. In some embodiments, the cable 105 is approximately one half of an inch wide. In some embodiments, the cable 105 is approximately one inch wide. In some embodiments, the cable 105 is approximately two inches wide. In some embodiments, the cable 105 is more than two inches wide.

In some embodiments, the system can include one, two, three, or more guides 106 on the chassis 102 of a trolley 100. In some embodiments, the guide 106 on the chassis 102 can guide the cable 105 toward guides 107 at the base 101 of the trolley 100. In some embodiments, the guide 106 on the chassis 102 can guide the cable 105 toward guides 108 on the legs 103 of the trolley 100. In some embodiments, the guide 106 on the chassis 102 of the trolley 100 is having an aperture facing upward. In some embodiments, having an aperture facing upward means the opening of the guide 106 is facing up, and thus the cable 105 can sit on top of the guide 106. In some embodiments, the guide 106 on the chassis 102 of the trolley 100 comprises a curved, semi-circular guide 500. In some embodiments, the guide 106 on the chassis 102 of the trolley 100 comprises a planar, semi-circular guide 400. In some embodiments, at least one guide 106 is able to be removed and repositioned on the trolley 100. In some embodiments, the cable 105 can be wrapped around the guide 106 to allow for storage of the trolley 100. The guide 106 can be a clip, a connector, a hook, or a clasp. The guide 106 can be C-shaped, L-shaped, or U-shaped. In some embodiments, the guide 106 has one portion attached to the trolley 100 and an aperture to receive the cable 105.

In some embodiments, the system can include one, two, three, or more guides on the base 101 of the trolley 100. In some embodiments, the guide 107 on the base 101 can guide the cable 105 toward guides 108 on the legs 103 of the trolley 100. In some embodiments, the guide 107 on the base 101 of the trolley 100 has an aperture facing downward. In some embodiments, having an aperture facing downward means the opening of the guide 107 is facing down, and thus the cable 105 can sit beneath the guide 107. In some embodiments, the guide 107 on the base 101 of the trolley 100 comprises a curved, semi-circular guide 500. In some embodiments, the guide 107 on the base 101 of the trolley 100 comprises a planar, semi-circular guide 400. In some embodiments, at least one guide 107 can be removed and repositioned on the trolley 100. In some embodiments, the cable 105 can be wrapped around the guide 107 to allow for storage of the trolley 100. The guide 107 can be a clip, a connector, a hook, or a clasp. The guide 107 can be C-shaped, L-shaped, or U-shaped. In some embodiments, the guide 107 has one portion attached to the trolley 100 and an aperture to receive the cable 105.

In some embodiments, the system can include guides 108 on the leg 103 of the trolley 100. In some embodiments, the system can include one, two, three, or more guides 108 on the leg 103 of the trolley 100. In some embodiments, the cable 105 is routed to the guides 108 on the leg 103 of the trolley 100 from the guide 106 on the chassis 102 of the trolley 100. In some embodiments, the cable 105 is routed to the guides 108 on the leg 103 of the trolley 100 from the guide 107 on the base 101 of the trolley 100. In some embodiments, the guides 108 on the leg 103 of the trolley 100 are located on the distal end of the leg 103. In some embodiments, the guides 108 are located approximately half an inch from the distal end of the leg 103. In some embodiments, the guides 108 are located approximately an inch from the distal end of the leg 103. In some embodiments, the guides 108 are located approximately two inches from the distal end of the leg 103. In some embodiments, the guides 108 are located approximately three inches from the distal end of the leg 103. In some embodiments, the guides 108 are located approximately three inches from the distal end of the leg 103. In some embodiments, the guides 108 are located approximately four inches from the distal end of the leg 103. In some embodiments, the guides 108 are located approximately five inches from the distal end of the leg 103. In some embodiments, two guides on the leg 103 of the trolley 100 have apertures facing upward. In some embodiments, having an aperture facing upward means the opening of the guide 108 is facing up, and thus the cable 105 is able to sit on top of the guide. In some embodiments, one guide 108 on the leg 103 of the trolley 100 has an aperture facing downward. In some embodiments, having an aperture facing downward means the opening of the guide 108 is facing down, and thus the cable 105 is able to sit beneath the guide. In some embodiments, the guides 108 on the leg 103 of the trolley 100 comprise planar, triangular guides 300. In some embodiments, the guides 108 are configured to route the cable 105 above one of the one or more wheels 104 of the trolley 100. In some embodiments, at least one guide 108 is able to be removed and repositioned on the trolley 100. In some embodiments, the cable 105 can be wrapped around the guides 108 to allow for storage of the trolley 100. The guides 108 can be clips, connectors, hooks, or clasps. The guides 108 can have various configurations, for example, the guides 108 can be C-shaped, L-shaped, or U-shaped. In some embodiments, the guides 108 each have one portion attached to the trolley 100 and an aperture to receive the cable 105.

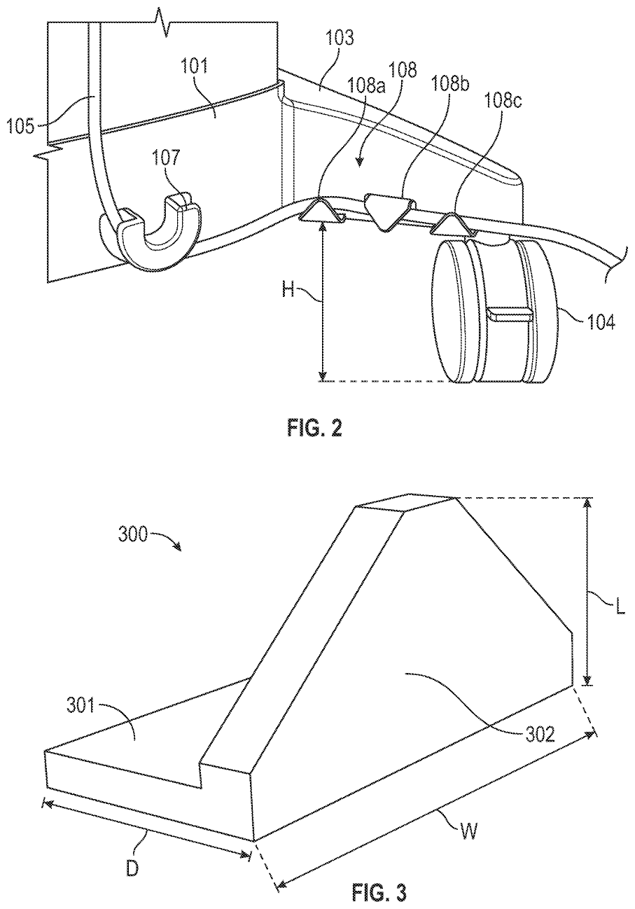

FIG. 2 illustrates an example of an embodiment of a base 101 and leg 103 of a trolley 100 using the systems, devices, and methods described herein. The guides 108 can guide the cable 105 above the wheel 104 of the trolley 100. In some embodiments, the guides 108 have apertures facing upward lift the cable 105 so that it does not touch the ground or wheel 104. In some embodiments, the guide 108 facing downward ensures that the cable 105 does not inadvertently fall off the guides 108 facing upward. In some embodiments, the guides 108 are static and in the open position. In some embodiments, the guides 108 can be opened and closed to keep the cable 105 stable. In some embodiments, the guides 108 are located on one leg 103 of the trolley 100. In some embodiments, the guides 108 are located on two legs 103 of the trolley 100. In some embodiments, the guides 108 are located on three legs 103 of the trolley 100. In some embodiments, the guides 108 are located on four legs 103 of the trolley 100. In some embodiments, the guides 108 are located on five legs 103 of the trolley 100. In some embodiments, the guides 108 are located on six legs 103 of the trolley 100. In some embodiments, the guides 108 are located on seven legs 103 of the trolley 100. In some embodiments, the guides 108 are located on eight legs 103 of the trolley 100. In some embodiments, the guides 108 are located on more than eight legs 103 of the trolley 100. In some embodiments, each guide 108 comprises a first portion that extends from the leg 103. In some embodiments, each guide 108 comprises a first portion that extends from the leg 103 and a second portion that extends from the first portion. In some embodiments, the first portion of each guide 108 is straight. In some embodiments, the first portion of each guide 108 is curved. In some embodiments, the second portion of each guide 108 is straight. In some embodiments, the second portion of each guide 108 is curved. In some embodiments, each guide 108 comprises more than two straight portions. In some embodiments, each guide 108 comprises more than two curved portions.

Referring to FIG. 3, L represents the length of the guides 108. In some embodiments, each guide 108 is approximately one twentieth of an inch tall. In some embodiments, each guide 108 is approximately one fifteenth of an inch tall. In some embodiments, each guide 108 is approximately one tenth of an inch tall. In some embodiments, each guide 108 is approximately one fifth of an inch tall. In an example of a preferred embodiment, each guide 108 is approximately one half of an inch tall. In some embodiments, each guide 108 is approximately one inch tall. In some embodiments, each guide 108 is approximately two inches tall. In some embodiments, each guide 108 is more than two inches tall. In FIG. 3, W represents the width of the guides 108. In some embodiments, each guide 108 is approximately one twentieth of an inch wide. In some embodiments, each guide 108 is approximately one fifteenth of an inch wide. In some embodiments, each guide 108 is approximately one tenth of an inch wide. In some embodiments, each guide 108 is approximately one fifth of an inch wide. In some embodiments, each guide 108 is approximately one half of an inch wide. In an example of a preferred embodiment, each guide 108 is approximately seventeen twentieths of an inch wide. In an example of a preferred embodiment, each guide 108 is approximately one inch wide. In some embodiments, each guide 108 is approximately two inches wide. In some embodiments, each guide 108 is more than two inches wide. In FIG. 3, D represents the depth of the guides 108. In some embodiments, each guide 108 has a depth of approximately one twentieth of an inch. In some embodiments, each guide 108 has a depth of approximately one fifteenth of an inch. In some embodiments, each guide 108 has a depth of approximately one tenth of an inch. In some embodiments, each guide 108 has a depth of approximately one fifth of an inch. In an example of a preferred embodiment, each guide 108 has a depth of approximately one half of an inch. In some embodiments, each guide 108 has a depth of approximately one inch. In some embodiments, each guide 108 has a depth of approximately two inches. In some embodiments, each guide 108 has a depth of approximately more than two inches.

In FIG. 2, H represents the height of the guides 108 above the floor. In some embodiments, the guides 108 are located approximately half an inch above the floor which the trolley 100 contacts. In some embodiments, the guides 108 are located approximately one inch above the floor which the trolley 100 contacts. In some embodiments, the guides 108 are located approximately one and a half inches above the floor which the trolley 100 contacts. In some embodiments, the guides 108 are located approximately two inches above the floor which the trolley 100 contacts. In some embodiments, the guides 108 are located approximately two and a half inches above the floor which the trolley 100 contacts. In an example of a preferred embodiment, the guides 108 are located approximately three inches above the floor which the trolley 100 contacts. In some embodiments, the guides 108 are located approximately three and a half inches above the floor which the trolley 100 contacts. In some embodiments, the guides 108 are located approximately four inches above the floor which the trolley 100 contacts. In some embodiments, the guides 108 are located approximately four and a half inches above the floor which the trolley 100 contacts. In some embodiments, the guides 108 are located approximately five inches above the floor which the trolley 100 contacts. In some embodiments, the guides 108 are located approximately five and a half inches above the floor which the trolley 100 contacts. In some embodiments, the guides 108 are located approximately six inches above the floor which the trolley 100 contacts. In some embodiments, the guides 108 are located approximately six and a half inches above the floor which the trolley 100 contacts. In some embodiments, the guides 108 are located approximately seven inches above the floor which the trolley 100 contacts. In some embodiments, the guides 108 are located approximately seven and a half inches above the floor which the trolley 100 contacts. In some embodiments, the guides 108 are located approximately eight inches above the floor which the trolley 100 contacts. In some embodiments, the guides 108 are located approximately eight and a half inches above the floor which the trolley 100 contacts. In some embodiments, the guides 108 are located approximately nine inches above the floor which the trolley 100 contacts. In some embodiments, the guides 108 are located approximately nine and a half inches above the floor which the trolley 100 contacts. In some embodiments, the guides 108 are located approximately ten inches above the floor which the trolley 100 contacts. In some embodiments, the guides 108 are located more than ten inches above the floor which the trolley 100 contacts.

In an example of a preferred embodiment, the guides 108 are located approximately one fifth of an inch above the wheel 104 of the trolley 100. In another example of preferred embodiment, the guides 108 are located approximately one quarter inch above the wheel 104 of the trolley 100. In another example of a preferred embodiment, the guides 108 are located approximately half an inch above the wheel 104 of the trolley 100. In some embodiments, the guides 108 are located approximately one inch above the wheel 104 of the trolley 100. In some embodiments, the guides 108 are located approximately one and a half inches above the wheel 104 of the trolley 100. In some embodiments, the guides 108 are located approximately two inches above the wheel 104 of the trolley 100. In some embodiments, the guides 108 are located approximately two and a half inches above the wheel 104 of the trolley 100. In some embodiments, the guides 108 are located approximately three inches above the wheel 104 of the trolley 100. In an example of a preferred embodiment, the guides 108 are located approximately three and one fifth inches above the floor which the trolley 100 contacts. In another example of a preferred embodiment, the guides 108 are located approximately three and a half inches above the floor which the trolley 100 contacts. In some embodiments, the guides 108 are located approximately four inches above the floor which the trolley 100 contacts. In some embodiments, the guides 108 are located approximately four and a half inches above the floor which the trolley 100 contacts. In some embodiments, the guides 108 are located approximately five inches above the floor which the trolley 100 contacts. In some embodiments, the guides 108 are located approximately five and a half inches above the floor which the trolley 100 contacts. In some embodiments, the guides 108 are located approximately six inches above the floor which the trolley 100 contacts. In some embodiments, the guides 108 are located approximately six and a half inches above the floor which the trolley 100 contacts. In some embodiments, the guides 108 are located approximately seven inches above the floor which the trolley 100 contacts. In some embodiments, the guides 108 are located approximately seven and a half inches above the floor which the trolley 100 contacts. In some embodiments, the guides 108 are located approximately eight inches above the floor which the trolley 100 contacts. In some embodiments, the guides 108 are located approximately eight and a half inches above the floor which the trolley 100 contacts. In some embodiments, the guides 108 are located approximately nine inches above the floor which the trolley 100 contacts. In some embodiments, the guides 108 are located approximately nine and a half inches above the floor which the trolley 100 contacts. In some embodiments, the guides 108 are located approximately ten inches above the floor which the trolley 100 contacts. In some embodiments, the guides 108 are located more than ten inches above the floor which the trolley 100 contacts.

In some embodiments, the guides 108c are located approximately half an inch from the distal end of the leg 103 of the trolley 100. In some embodiments, the guides 108c are located approximately one inch from the distal end of the leg 103 of the trolley 100. In some embodiments, the guides 108c are located approximately one and a half inches from the distal end of the leg 103 of the trolley 100. In some embodiments, the guides 108c are located approximately two inches from the distal end of the leg 103 of the trolley 100. In an example of a preferred embodiment, the guides 108c are located approximately two and a half inches from the distal end of the leg 103 of the trolley 100. In an example of a preferred embodiment, the guides 108c are located approximately two and four fifths inches from the distal end of the leg 103 of the trolley 100. In another example of a preferred embodiment, the guides 108c are located approximately three inches from the distal end of the leg 103 of the trolley 100. In some embodiments, the guides 108c are located approximately three and a half inches from the distal end of the leg 103 of the trolley 100. In some embodiments, the guides 108c are located approximately four inches from the distal end of the leg 103 of the trolley 100. In some embodiments, the guides 108c are located approximately four and a half inches from the distal end of the leg 103 of the trolley 100. In some embodiments, the guides 108c are located approximately five inches from the distal end of the leg 103 of the trolley 100. In some embodiments, the guides 108c are located approximately five and a half inches from the distal end of the leg 103 of the trolley 100. In some embodiments, the guides 108c are located approximately six inches from the distal end of the leg 103 of the trolley 100. In some embodiments, the guides 108c are located approximately six and a half inches from the distal end of the leg 103 of the trolley 100. In some embodiments, the guides 108c are located approximately seven inches from the distal end of the leg 103 of the trolley 100. In some embodiments, the guides 108c are located approximately seven and a half inches from the distal end of the leg 103 of the trolley 100. In some embodiments, the guides 108c are located approximately eight inches from the distal end of the leg 103 of the trolley 100. In some embodiments, the guides 108c are located approximately eight and a half inches from the distal end of the leg 103 of the trolley 100. In some embodiments, the guides 108c are located approximately nine inches from the distal end of the leg 103 of the trolley 100. In some embodiments, the guides 108c are located approximately nine and a half inches from the distal end of the leg 103 of the trolley 100. In some embodiments, the guides 108c are located approximately ten inches from the distal end of the leg 103 of the trolley 100. In some embodiments, the guides 108c are located more than ten inches from the distal end of the leg 103 of the trolley 100. In some embodiments,

In some embodiments, the guide 108a on the portion of the leg 103 nearest the base 101 of the trolley 100 lifts the cable 105 above floor which the trolley 100 contacts. In some embodiments, the guide 108a has an aperture facing upward. In some embodiments, the guide 108b nearest the center of the leg 103 of the trolley 100 keeps the cable 105 secured on the guide 108c. In some embodiments, the guide 108b has an aperture facing downward. In some embodiments, the guide 108c nearest the distal end of the leg 103 of the trolley 100 lifts the cable 105 above the wheel 104. In some embodiments, the guide 108c has an aperture facing upward. In some embodiments, the cable 105 is positioned through the apertures of the guide 108a, the guide 108b, and the guide 108c.

FIG. 3 illustrates an example of an embodiment of a planar, triangular guide 300 used in the systems, devices, and methods described herein. In some embodiments, a planar, triangular guide 300 comprises a substantially planar body 301 extending from the trolley 100. In some embodiments, a planar, triangular guide 300 comprises a triangular body 302 extending approximately perpendicularly from a distal end of the substantially planar body 301. In some embodiments, planar, triangular guides 300 hold the cable 105 in place, directing it in a substantially straight line. In some embodiments, the guide 106 on the chassis 102 is a planar, triangular guide 300. In some embodiments, the guide 107 on the base 101 is a planar, triangular guide 300. In some embodiments, the guides 108 on the leg 103 are planar, triangular guides 300. In FIG. 3, L represents the length of the guides 108. In FIG. 3, W represents the width of the guides 108. In FIG. 3, D represents the depth of the guides 108.

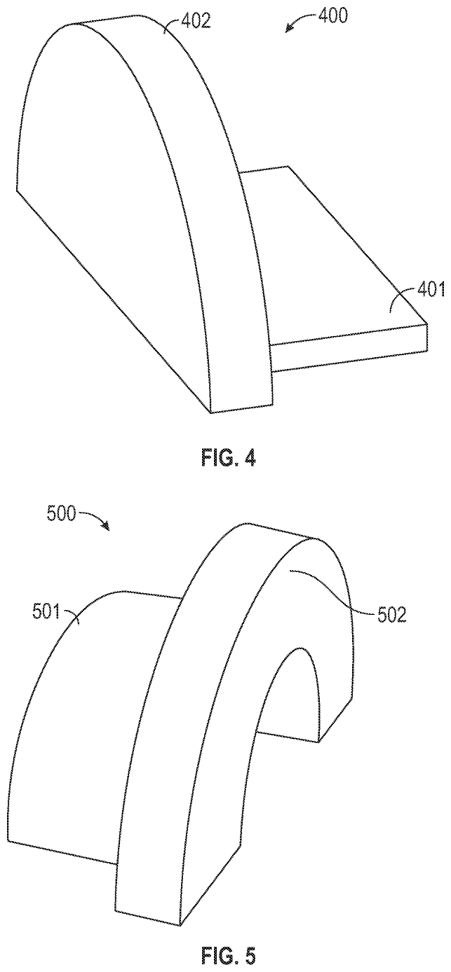

FIG. 4 illustrates an example of an embodiment of a planar, semi-circular guide 400 used in the systems, devices, and methods described herein. In some embodiments, a planar, semi-circular guide 400 comprises a substantially planar body 401 extending from the trolley 100. In some embodiments, a planar, semi-circular guide 400 comprises a semi-circular body 402 extending approximately perpendicularly from a distal end of the substantially planar body 401. In some embodiments, planar, semi-circular guides 400 hold the cable 105 in place, directing it in a substantially straight line. In some embodiments, the guide 106 on the chassis 102 is a planar, semi-circular guide 400. In some embodiments, the guide 107 on the base 101 is a planar, semi-circular guide 400. In some embodiments, the guides 108 on the leg 103 are planar, semi-circular guides 400.

FIG. 5 illustrates an example of an embodiment of a curved, semi-circular guide 500 used in the systems, devices, and methods described herein. In some embodiments, a curved, semi-circular guide 500 comprises a substantially curved body 501 extending from the trolley 100. In some embodiments, a curved, semi-circular guide 500 comprises a semi-circular body 502 extending approximately perpendicularly from a distal end of the substantially curved body 501. In some embodiments, a curved, semi-circular guide 500 is fixed to the base 101 of the trolley 100 to allow the cable 105 to be routed to a position 701 along one leg 103 or to a position 801 along another leg 103. In some embodiments, a curved, semi-circular guide 500 is fixed to the chassis 102 of the trolley 100 to allow the cable 105 to be routed to a position 701 along one leg 103 or to a position 801 along another leg 103. In some embodiments, the guide 106 on the chassis 102 is a curved, semi-circular guide 500. In some embodiments, the guide 107 on the base 101 is a curved, semi-circular guide 500. In some embodiments, the guides 108 on the leg 103 are curved, semi-circular guides 500.

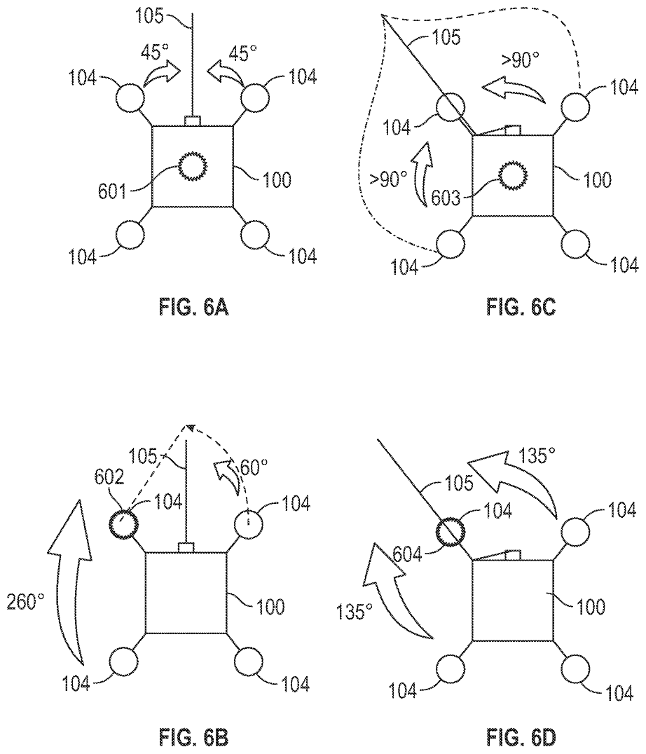

FIG. 6A illustrates an example of an embodiment of the rotation of a trolley 100 about its center without using the systems, devices, and methods described herein. In some embodiments, at 601, the trolley 100 can rotate approximately 45 degrees about the center of the trolley 100 without the system described herein. In some embodiments, when the trolley 100 rotates more than approximately 45 degrees about its center without using the systems, devices, and methods described herein, the cable 105 may interfere with the movement of a wheel 104.

FIG. 6B illustrates an example of an embodiment of the rotation of a trolley 100 about its wheel 104 without using the systems, devices, and methods described herein. In some embodiments, at 602, the trolley 100 can rotate approximately 60 degrees about the wheel 104 of the trolley 100 without the system described herein. In some embodiments, when the trolley 100 rotates more than approximately 60 degrees about its wheel 104 without using the systems, devices, and methods described herein, the cable 105 may interfere with the movement of a wheel 104.

FIG. 6C illustrates an example of an embodiment of the rotation of a trolley 100 about its center using the systems, devices, and methods described herein. In some embodiments, at 603, the trolley 100 can rotate greater than approximately 90 degrees about the center of the trolley 100 with the system described herein.

FIG. 6D illustrates an example of an embodiment of the rotation of a trolley 100 about its wheel 104 using the systems, devices, and methods described herein. In some embodiments, at 604, the trolley 100 can rotate approximately 135 degrees about the wheel 104 of the trolley 100 with the system described herein. In some embodiments, when the trolley 100 rotates more than approximately 135 degrees about its wheel 104 using the systems, devices, and methods described herein, the cable 105 may interfere with the movement of a wheel 104.

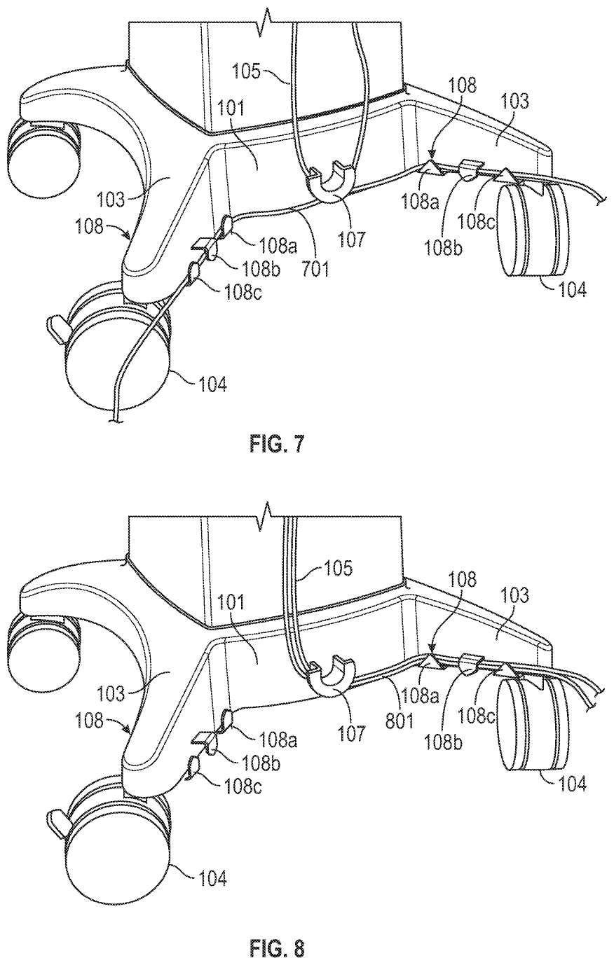

FIG. 7 illustrates an example of an embodiment of a possible cable position 701 using systems, devices, and methods described herein. In some embodiments, the cable 105 can be routed in a position 701 along a leg 103 of the trolley 100. In some embodiments, the cable 105 can be repositioned from position 801 to position 701. In some embodiments, rerouting the cable 105 to position 701 allows an increased range of motion in the other direction. In some embodiments, additional routes entail routing the cable 105 along other legs 103 of the trolley 100.

FIG. 8 illustrates an example of an embodiment of an alternate possible cable position 801 using systems, devices, and methods described herein. In some embodiments, the cable 105 can be routed at a position 801 along a leg 103 of the trolley 100. In some embodiments, the cable 105 can be repositioned from position 701 to position 801. In some embodiments, rerouting the cable 105 to position 801 allows an increased range of motion in the other direction. In some embodiments, additional routes entail routing the cable 105 along other legs 103 of the trolley 100.

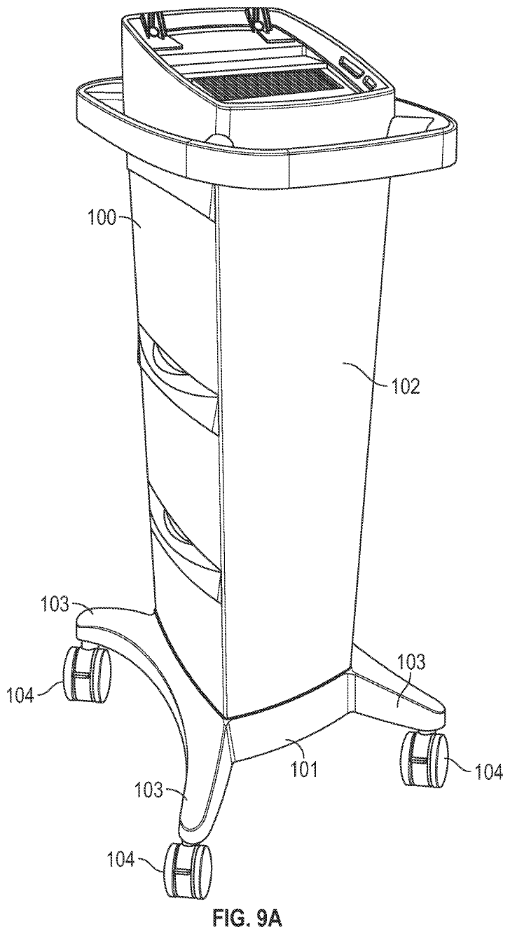

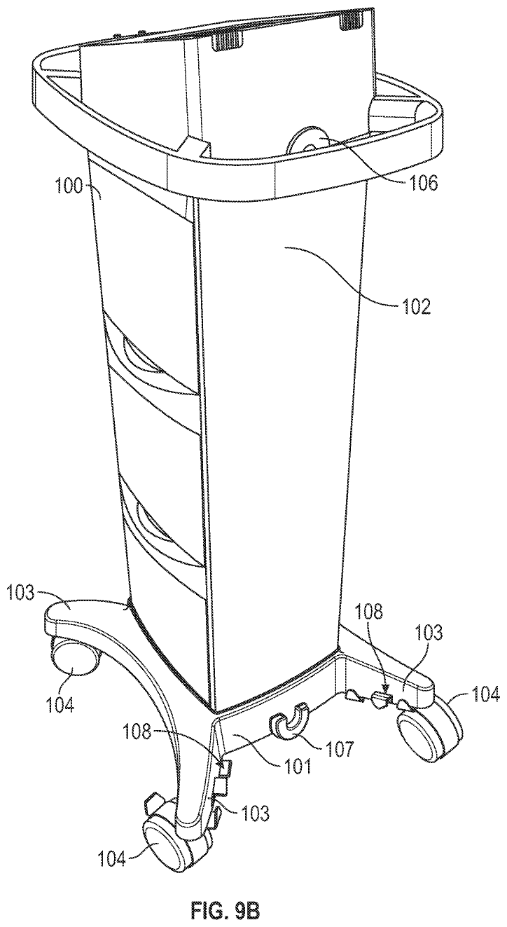

FIG. 9A illustrates an example of an embodiment of a front view of the trolley 100. In some embodiments, the trolley 100 comprises a medical trolley. In some embodiments, a medical trolley can mean a movable cart used in hospitals. In some embodiments, a medical trolley can mean a movable cart used for medical purposes. In some embodiments, the trolley 100 comprises a commercial trolley. In some embodiments, a commercial trolley can mean a movable cart used in a commercial office. In some embodiments, a commercial trolley can mean a movable cart used for commercial purposes. As illustrated in FIG. 9A, in some embodiments, the trolley 100 includes: a base 101, a chassis 102 fixed to the top of the base 101, legs 103 extending from the base 101, and wheels 104 fixed to the bottom of the legs 103. In some embodiments, the base 101 of the trolley 100 is configured to support the trolley 100. In some embodiments, the base 101 of the trolley 100 is fixed below the chassis 102 of the trolley 100. In some embodiments, the base 101 of the trolley 100 is fixed to the legs 103 of the trolley. In some embodiments, the base 101 of the trolley 100 is fixed to the wheels of the trolley 100.

FIG. 9B illustrates an example of an embodiment of a back view of the trolley 100. In some embodiments, the trolley 100 comprises a medical trolley. In some embodiments, a medical trolley can mean a movable cart used in hospitals. In some embodiments, a medical trolley can mean a movable cart used for medical purposes. In some embodiments, the trolley 100 comprises a commercial trolley. In some embodiments, a commercial trolley can mean a movable cart used in a commercial office. In some embodiments, a commercial trolley can mean a movable cart used for commercial purposes. As illustrated in FIG. 9B, in some embodiments, the trolley 100 includes: a base 101, a chassis 102 fixed to the top of the base 101, legs 103 extending from the base 101, wheels 104 fixed to the bottom of the legs 103, a guide 106 on the chassis 102, a guide 107 on the base 101, and guides 108 on the legs 103. In some embodiments, the base 101 of the trolley 100 is configured to support the trolley 100. In some embodiments, the base 101 of the trolley 100 is fixed below the chassis 102 of the trolley 100. In some embodiments, the base 101 of the trolley 100 is fixed to the legs 103 of the trolley. In some embodiments, the base 101 of the trolley 100 is fixed to the wheels of the trolley 100.

Unless a term is expressly defined in this disclosure or the context dictates a certain interpretation to possess a described meaning, there is no intent to limit the meaning of such term, either expressly or indirectly, beyond its plain or ordinary meaning. Any element or feature in a claim that does not explicitly state “means for” performing a specified function, or “step for” performing a specific function, is not to be interpreted as a “means” or “step” clause as specified in 35 U.S.C. § 112, paragraph 6.

The following is a list of certain components that are described and enumerated in this disclosure in reference to the above-listed figures. However, any aspect of the devices illustrated in the figures, whether or not named out separately herein, can form a portion of various embodiments of the systems, devices, and methods described herein and may provide basis for claim limitation relating to such aspects, with or without additional description. The enumerated components include:
100 Trolley with a guide for a cable
101 The base of the trolley
102 The chassis of the trolley
103 A leg of the trolley
104 A wheel of the trolley
105 The cable of the trolley
106 A guide on the chassis of a trolley
107 A guide on the base of a trolley
108 Guides on the leg of a trolley
108a The guide on the leg nearest the base of a trolley
108b The guide nearest the center of the leg of a trolley
108c The guide nearest the distal end of the leg of a trolley
109 The device placed on the trolley
300 A planar, triangular guide
301 A planar body of a planar, triangular guide
302 A triangular body of a planar, triangular guide
400 A planar, semi-circular guide
401 A planar body of a planar, semi-circular guide
402 A semi-circular body of a planar, semi-circular guide
500 A curved, semi-circular guide
501 A curved body of a curved, semi-circular guide
502 A semi-circular body of a curved, semi-circular guide
601 Rotation of trolley about center without guide
602 Rotation of trolley about wheel without guide
603 Rotation of trolley about center with guide
604 Rotation of trolley about wheel with guide
701 Potential cable position
801 Alternate potential cable position

### Example Embodiments

Embodiment 1. A system comprising: a trolley comprising: a base configured to support the trolley; a chassis fixed above the base of the trolley; one or more legs extending from the base of the trolley; and one or more wheels fixed beneath the one or more legs of the trolley; a cable connected to the trolley; and at least one guide fixed to one of the one or more legs of the trolley, wherein: the at least one guide is configured to route the cable above one of the one or more wheels of the trolley.

Embodiment 2. The system of Embodiment 1, wherein the trolley comprises a medical trolley.

Embodiment 3. The system according to any one of Embodiment 1-2, wherein the at least one guide comprises: a substantially planar body extending from the trolley; and a triangular body extending approximately perpendicularly from a distal end of the substantially planar body.

Embodiment 4. The system according to any one of Embodiment 1-2, wherein the at least one guide comprises: a substantially planar body extending from the trolley; and a semi-circular body extending approximately perpendicularly from a distal end of the substantially planar body.

Embodiment 5. The system according to any one of Embodiment 1-2, wherein the at least one guide comprises: a substantially curved body extending from the trolley; and a semi-circular body extending approximately perpendicularly from a distal end of the substantially curved body.

Embodiment 6. The system according to any one of Embodiment 1-5, wherein the at least one guide is able to be removed and repositioned on the trolley.

Embodiment 7. The system according to any one of Embodiment 1-5, wherein the at least one guide is located on one of the one or more legs of the trolley.

Embodiment 8. The system according to any one of Embodiment 1-5, wherein: the trolley has at least two legs; at least one guide is located on each of the at least two legs of the trolley; and the cable can be repositioned from one of the at least two legs of the trolley to another of the at least two legs of the trolley.

Embodiment 9. The system according to any one of Embodiment 1-5, further comprising: a guide having an aperture facing downward on the base of the trolley; two guides having apertures facing upward on one of the one or more legs of the trolley; and one guide having an aperture facing downward on one of the one or more legs of the trolley.

Embodiment 10. The system of Embodiment 9, further comprising a guide on the chassis of the trolley.

Embodiment 11. A system for guiding a cable, comprising: at least one guide fixed to a trolley, wherein the at least one guide is configured such that positioning a cable of the trolley along the at least one guide routes the cable above a wheel of the trolley.

Embodiment 12. The system of Embodiment 11, wherein the trolley comprises a medical trolley.

Embodiment 13. The system according to any one of Embodiment 11-12, wherein the at least one guide comprises: a substantially planar body extending from the trolley; and a triangular body extending approximately perpendicularly from a distal end of the substantially planar body.

Embodiment 14. The system according to any one of Embodiment 11-12, wherein the at least one guide comprises: a substantially planar body extending from the trolley; and a semi-circular body extending approximately perpendicularly from a distal end of the substantially planar body.

Embodiment 15. The system according to any one of Embodiment 11-12, wherein the at least one guide comprises: a substantially curved body extending from the trolley; and a semi-circular body extending approximately perpendicularly from a distal end of the substantially curved body.

Embodiment 16. The system according to any one of Embodiment 11-15, wherein the at least one guide is able to be removed and repositioned on the trolley.

Embodiment 17. The system according to any one of Embodiment 11-15, wherein the at least one guide is located on one or more legs of the trolley.

Embodiment 18. The system according to any one of Embodiment 11-15, wherein: the trolley has at least two legs; at least one guide is located on each of the at least two legs of the trolley; and the cable can be repositioned from one of the at least two legs of the trolley to another of the at least two legs of the trolley.

Embodiment 19. The system according to any one of Embodiment 11-15, further comprising: a guide having an aperture facing downward on a base of the trolley; two guides having apertures facing upward on a leg of the trolley; and one guide having an aperture facing downward on the leg of the trolley.

Embodiment 20. The system of Embodiment 19, further comprising a guide on a chassis of the trolley.

### IMPLEMENTATION DETAILS

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached, or coupled to the other feature or element or intervening features or elements may be present. In contrast, when a feature or element is referred to as being “directly connected,” “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a,” “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/.”

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together. B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly,” “downwardly,” “vertical,” “horizontal” and the like may be used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing numeric values of magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

1. A system comprising:
a trolley comprising:
a base configured to support the trolley;
a chassis fixed above the base of the trolley;
one or more legs extending from the base of the trolley; and
one or more wheels fixed beneath the one or more legs of the trolley;
a cable connected to the trolley; and
at least one guide fixed to one of the one or more legs of the trolley, wherein:
the at least one guide is configured to route the cable above one of the one or more wheels of the trolley.

2. The system of claim 1, wherein the trolley comprises a medical trolley.

3. The system of claim 1, wherein the at least one guide comprises:
a substantially planar body extending from the trolley; and
a triangular body extending approximately perpendicularly from a distal end of the substantially planar body.

4. The system of claim 1, wherein the at least one guide comprises:
a substantially planar body extending from the trolley; and
a semi-circular body extending approximately perpendicularly from a distal end of the substantially planar body.

5. The system of claim 1, wherein the at least one guide comprises:
a substantially curved body extending from the trolley; and
a semi-circular body extending approximately perpendicularly from a distal end of the substantially curved body.

6. The system of claim 1, wherein the at least one guide is able to be removed and repositioned on the trolley.

7. The system of claim 1, wherein the at least one guide is located on one of the one or more legs of the trolley.

8. The system of claim 1, wherein:
the trolley has at least two legs;
at least one guide is located on each of the at least two legs of the trolley; and
the cable can be repositioned from one of the at least two legs of the trolley to another of the at least two legs of the trolley.

9. The system of claim 1, further comprising:
a guide having an aperture facing downward on the base of the trolley;
two guides having apertures facing upward on one of the one or more legs of the trolley; and
one guide having an aperture facing downward on one of the one or more legs of the trolley.

10. The system of claim 9, further comprising a guide on the chassis of the trolley.

11. A system for guiding a cable, comprising:
at least one guide fixed to a trolley, wherein the at least one guide is configured such that positioning a cable of the trolley along the at least one guide routes the cable above a wheel of the trolley.

12. The system of claim 11, wherein the trolley comprises a medical trolley.

13. The system of claim 11, wherein the at least one guide comprises:
a substantially planar body extending from the trolley; and
a triangular body extending approximately perpendicularly from a distal end of the substantially planar body.

14. The system of claim 11, wherein the at least one guide comprises:
a substantially planar body extending from the trolley; and
a semi-circular body extending approximately perpendicularly from a distal end of the substantially planar body.

15. The system of claim 11, wherein the at least one guide comprises:
a substantially curved body extending from the trolley; and
a semi-circular body extending approximately perpendicularly from a distal end of the substantially curved body.

16. The system of claim 11, wherein the at least one guide is able to be removed and repositioned on the trolley.

17. The system of claim 11, wherein the at least one guide is located on one or more legs of the trolley.

18. The system of claim 11, wherein:
the trolley has at least two legs;
at least one guide is located on each of the at least two legs of the trolley; and
the cable can be repositioned from one of the at least two legs of the trolley to another of the at least two legs of the trolley.

19. The system of claim 11, further comprising:
a guide having an aperture facing downward on a base of the trolley;
two guides having apertures facing upward on a leg of the trolley; and
one guide having an aperture facing downward on the leg of the trolley.

20. The system of claim 19, further comprising a guide on a chassis of the trolley.
